(19) United States
(12) Patent Application Publication
(10) Pub. No.: US 2009/0182225 A1
(43) Pub. Date: Jul. 16, 2009

(54) Safety Marked Fibers and Catheters

(75) Inventors: Brian Foley, Wilbraham, MA (US); Joe Mooney, East Longmeadow, MA (US)

Correspondence Address: BOLESH J. SKUTNIK;CERAMOPTEC INDUSTRIES, INC., 515 SHAKER RD., EAST LONGMEADOW, MA, 01028, US

(73) Assignee: CeramOptec Industries Inc.

(21) Appl. No.: 12/348,816

(22) Filed: Jan. 5, 2009

Related U.S. Application Data
(60) Provisional application No. 61/009,867, filed on Jan. 3, 2008.

(51) Int. Cl. A61B 18/20

(52) U.S. Cl. 600/424; 606/16

(57) ABSTRACT

A device and method are presented that allow for accurate insertion, positioning and withdrawal of a laser fiber/catheter for minimally invasive laser treatment by utilizing a single mark in the form of a continuous spiral on either a fiber jacket or a catheter (introducer) or both. Viewed from a fixed position the frequency of the appearance of the line can be used to regulate the speed of withdrawal either manually, automatically or through a combination of both, i.e., visual speed feedback and manual withdrawal, automatic speed feedback and manual pullback or automatic feedback and withdrawal. The distal spiral end terminates preferentially in a circumferential loop at some distance back from the distal tip of the catheter or fiber jacket to serve as a safety marker to indicate when to stop lasing during withdrawal to prevent accidental exposure of the patient or attending hospital personnel. Additionally, approaching the distal end, the spiral line can change in color, hue, thickness or some other feature to better provide advanced warning of the approaching fiber end

### DOMESTIC PRIORITY UNDER 35 USC 119(e)

This application claims the benefit of U.S. Provisional Application Ser. No. 61/009,867 filed Jan. 3, 2008, entitled “Safety Marked Fibers and Catheters” by Brian Foley and Joe Mooney, which is incorporated by reference herein.

### BACKGROUND OF THE INVENTION

1. Field of the Invention

The present invention is related to safe and effective use of medical laser fibers and catheters as delivery systems for medical laser energy. More particularly the invention relates to devices marked to facilitate determining fiber and/or catheter position within a patient's body during laser treatment involving withdrawal of the delivery system.

2. Information Disclosure Statement

In certain invasive medical procedures thermal or other energy is administered to a patient with beneficial effects. For example, energy can be used to detect a tumor or a region of the body, or to destroy or denature diseased or malfunctioning body tissue. Other types of medical treatment utilize laser energy, for example, laser energy is delivered to the inner wall of a vein where it is absorbed, restructuring the vein. These procedures require durable optical fibers that distribute light in a predictable and controlled manner.

Laser energy generally is delivered to an interior area of a patient's body by means of an optical fiber. However, the tips of such optical fibers could cause trauma to soft tissues, if the tip accidentally touches them. Therefore, for many invasive applications it is desirable to insert the optical fiber into a body very carefully. The optical fiber is, thus typically inserted into a flexible introducer sheath which acts to guide and protect the optical fiber—and the surrounding tissue—while it is being inserted into the region of the body to be treated. An introducer sheath is frequently inserted into body tissue over a guide wire and then the optical fiber can be inserted once the introducer sheath is in place in the body and usually the guide wire is removed. Precision and control are important to produce positive clinical outcomes; however, there is significant difficulty in determining fiber position and depth.

Usually the introducer sheath is positioned so that the optical fiber protrudes a few centimeters from the end of the introducer sheath to deliver laser energy efficiently from the tip of the fiber to the surrounding tissue.

Ultrasound and/or direct visualization of an aiming laser beam from the optical fiber through the skin to detect optical fiber tip protrusion from the introducer sheath are well known in the art. However, these methods are unsatisfactory for a number of reasons. The use of ultrasound requires additional equipment which is expensive and complicates the procedure, especially, as often expert interpretation of the images is required. Visualization of the laser beam is imprecise and at best only a guide and then only in regions of the body which are sufficiently close to the surface of the skin. Therefore, conventional techniques are not suitable for use in a wide range of applications.

Furthermore, as a result of it being difficult for an operator or surgeon to determine when the distal end of the optical fiber is approaching the distal end of the introducer sheath, damage can readily occur to the soft tissues if the optical fiber is initially accidentally extended too far beyond the introducer sheath. As a result, an operator must insert the optical fiber very slowly into the introducer sheath and must proceed cautiously at all times, which makes the procedure slow.

U.S. Pat. Nos. 6,986,766 and 6,981,971 by Caldera et al. disclose optical fibers with separate markers to aid optical fiber alignment. The most distal marker indicates the position where the optical fiber tip is substantially in alignment with the distal end of the introducer sheath. The most proximal marker indicates the distance the introducer sheath should be withdrawn to allow exposure of the fiber tip beyond the introducer sheath of a predetermined length. The markings allow the operator to safely position the fiber tip and quickly expose a predetermined length of the fiber beyond the introducer sheath. Graduated markings on the sheath allow the operator to withdraw the introducer sheath at a defined distance per unit of time or distance per laser pulse, if a pulsed laser is used. However, this fails to provide the operator feedback during introducer sheath withdrawal. The separate markings disclosed by Caldera et al. require the operator to remember the last marking passed and anticipate the distance until the next marker is exposed. Continuous feedback to the operator would allow the operator to know the position of the introducer sheath relative to the optical fiber tip at any particular instant.

U.S. Pub. No. US 2004/0034342 A1 by Barton et al. disclose an optical fiber for surgical use with non-alphanumeric markings. The markings are designed to indicate to the operator, fiber depth of a surgical instrument into tissue. Three or more non-alphanumeric characters mark a treatment region, a first depth indicating region, and a second depth indicating region. The operator views at least two non-alphanumeric markings on the surgical instrument which are markedly different from other markings on the instrument. These markings will indicate the depth of the instrument into tissue. Once again, Barton et al. fail to provide the operator continuous feedback during fiber use. The separate markings disclosed require the operator to remember the last marking passed and anticipate the distance until the next marker is exposed. Continuous feedback to the operator would allow the operator to know the position of the surgical instrument at any particular instant.

U.S. Pat. No. 5,638,483 by Konwitz discloses markings for a side-emitting optical fiber. A marking extends approximately 180° around the circumference of the fiber with a discernable center and edges. The marking allows the operator to view the marking in all orientations of the fiber where the radiation exit region faces away from the user. The disclosed marking by Konwitz allows the operator to determine the orientation of the fiber, but it fails to indicate fiber depth into tissue. Moreover, the marking is particularly appropriate for a side-emitting fiber where orientation is important to indicate the direction of the emitted radiation. However, orientation is less critical with straight-emitting fibers because radiation direction is easily discernable.

There is thus a need for an optical fiber treatment system that improves on the state of the art by indicating optical fiber depth to the operator by providing continuous feedback. This prevents the operator from guessing the distance between graduated markings and estimating fiber withdrawal rates. This invention addresses these needs.

### OBJECTIVES AND BRIEF SUMMARY OF THE INVENTION

It is an objective of this invention to provide an improved device for safe, effective minimally invasive laser treatment.

It is also an objective of this invention to provide a means for determining laser fiber position with respect to the introduction catheter, when present.

It is another objective of this invention to provide a marking system for determining laser fiber/catheter position within the body during laser treatment.

It is still another objective of this invention to facilitate manual regulation of pull-back speed of a laser fiber/catheter through direct visualization or an automatic reader device.

It is yet another objective of this invention to allow automatic withdrawal and feedback of laser fiber/catheter pull-back speed during laser treatment.

It is still another objective of this invention to provide a means to more safely complete a treatment while minimizing accidental exposure of the patient or attending hospital personnel to the active laser beam.

Briefly stated, the present invention provides a device and method that allow for accurate insertion, positioning and withdrawal of a laser fiber/catheter for minimally invasive laser treatment by utilizing a single mark in the form of a continuous spiral on either an optical fiber, optical fiber jacket, a catheter (introducer) or both catheter and fiber/fiber jacket Viewed from a fixed position the frequency of the appearance of the line can be used to regulate the speed of withdrawal either manually, automatically, or through a combination of both, i.e., visual speed feedback and manual withdrawal, automatic speed feedback and manual withdrawal or automatic feedback and withdrawal. The distal spiral end terminates preferentially in a circumferential loop at some distance back from the distal tip of the catheter or fiber jacket to serve as a safety marker to indicate when to stop lasing during withdrawal to prevent accidental exposure of the patient or attending hospital personnel. Approaching the distal end the spiral line can change in color, hue, thickness or some other feature to better provide advanced warning of the approaching fiber end.

The above and other objects, features and advantages of the present invention will become apparent from the following description read in conjunction with the accompanying drawings, (in which like reference numbers in different drawings designate the same elements).

### BRIEF DESCRIPTION OF FIGURES

FIG. 1a shows a plane view of a preferred embodiment of the invention in which a spiral pattern varying in thickness is printed on the introducer sheath.

FIG. 1b shows a plane view of the side fiber indicated in FIG. 1a emphasizing two marks for assessing correct placement.

FIG. 2 shows a plane view of a preferred embodiment of the invention in which a spiral pattern varying in thickness is printed on the optical fiber.

FIG. 3 shows another variation of a preferred embodiment in which spiral pattern increases its frequency when approaching distal end.

FIG. 4 shows another preferred embodiment in which a sensor is placed to give the surgeon position feedback of the catheter/fiber ensemble.

FIG. 5 shows another preferred embodiment in which a sensor is placed to give position information to an automatic pull-back device.

### DETAILED DESCRIPTION OF PREFERRED EMBODIMENTS

The present invention provides a device and method that allow for accurate insertion, positioning and withdrawal of a laser fiber/catheter for minimally invasive laser treatment by utilizing a single mark in the form of a continuous spiral on either a fiber jacket or a catheter (introducer) or on both and additionally position marks may be printed on the optical fiber.

The spiral mark is used in order to improve insertion and withdrawal control during laser treatment. Depending on the treatment, it can be printed directly on the optical fiber or on the catheter in which fiber is inserted.

In the case that fiber is used without catheter guide, spiral and position markings are both printed on the fiber. Depending on the treatment, the physician may introduce the fiber directly (e.g., in tracheal/esophagus treatment) or through a previously inserted needle (e.g., greater saphenous vein treatment). In both cases, position marks are helpful to precisely and safely determine fiber placement, so a physician can be assured that optical fiber will not exceed treatment area limits. When treatment starts, physician withdraws fiber while lasing. Pullback speed can be important in the treatment's outcome: if it is too fast, laser effects can be insufficient to cause the desired effect on the tissue; if it is too slow, tissue may be unnecessarily damaged.

When the optical fiber is introduced through a catheter guide, spiral mark is printed on the catheter and position marks are printed on the fiber. Physician first introduces catheter and then the fiber through it. Fiber placement relative to catheter is assessed by the position marks, so physician can be assured that optical fiber tip will protrude catheter by an appropriate distance.

When treatment starts, physician withdraws fiber/catheter while lasing. Since pullback speed can be important in the treatment's outcome, spiral properties can be used as feedback to precisely and safely control of fiber/catheter withdrawal, either manually or automatically. Furthermore, spiral properties can be useful to determine when to stop lasing during withdrawal to prevent accidental exposure of the patient or attending hospital personnel.

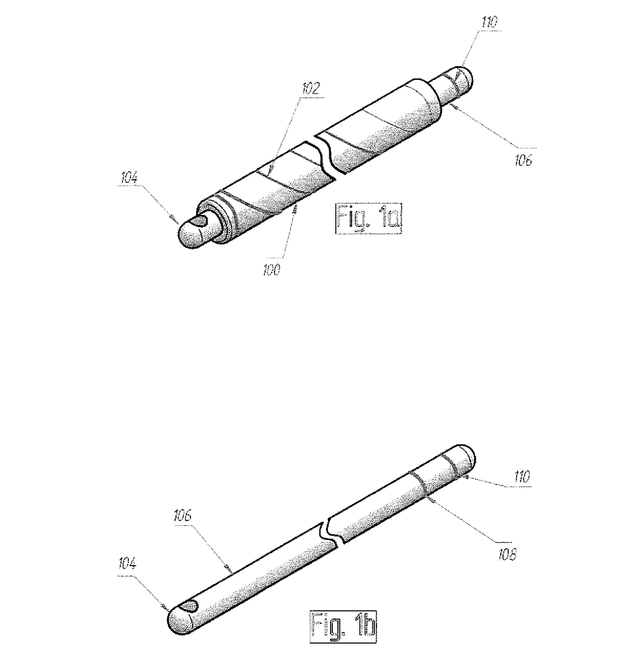

A preferred embodiment is shown in FIGS. 1a and 1b. Pattern 102 is printed along the length of laser introducer 100 in the form of a spiral. The surgeon inserts introducer 100 into a vein to be treated or alternate treatment area over a guide wire and then the optical fiber 106 can be inserted once the introducer sheath 100 is in place in the body and the guide wire is removed. The fiber has at least two position marks at its proximal end. One of the position marks 108 corresponds to the position where fiber tip 104 coincides or aligns with the distal end of introducer 100. The other position mark 110 indicates the desired position of fiber tip 104 with respect to introducer 100 to start lasing. Once fiber 106 is in place with its tip 104 exposed a few centimeters, fiber and sheath are locked together so that energized fiber tip 104 does not accidentally enter the sheath 100 while lasing. When the surgeon pulls the fiber/catheter ensemble back while applying energy to the inner walls of the vein or alternate treatment area, he is able to use spiral pattern 102 as a guide for knowing the relative position of the fiber/catheter as well as for moving it outward at a determined speed. The thickness or broadness of spiral 102 increases as it approaches the distal end and is an indicator of closeness of fiber tip 104 to the vein's opening at the skin. This helps to avoid accidental exposure of the patient's skin at the incision or elsewhere to the full power of the laser beam being used to close the vein. Also attending hospital personnel, such as nurses, surgeons, anesthesiologists, are protected from exposure to the laser beam energy.

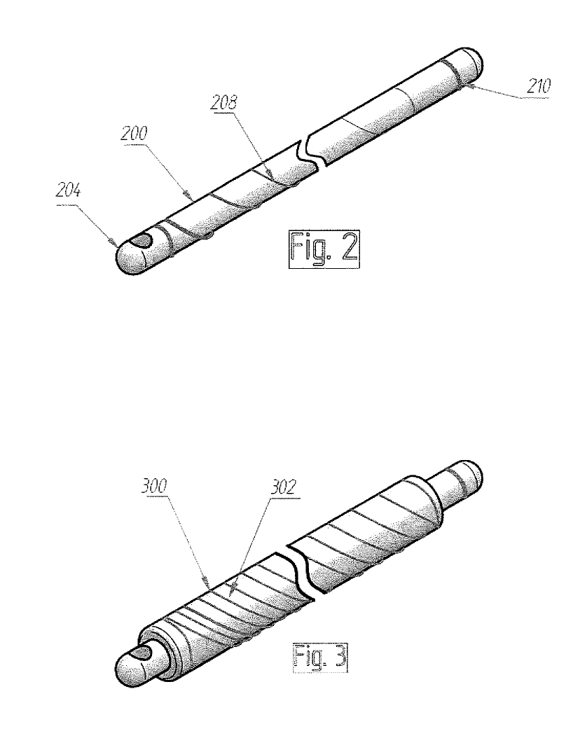

In another preferred embodiment, shown in FIG. 2, pattern 208 is printed along the length of optical fiber 200 in the form of a spiral. The physician inserts optical fiber 200 into the vein to be treated. The fiber has a mark 210 at its proximal end. When fiber mark 210 reaches patient's skin, it corresponds to the position where fiber tip 204 coincides with the distal extreme of the vein to be treated. When the physician pulls the fiber back while applying energy to the inner walls of the vein, he is able to use spiral pattern 208 as a guide for knowing the relative position of the fiber as well as for moving it outward at a determined speed. The thickness of spiral 208 increases as it approaches the distal end and is an indicator of closeness of fiber tip 204 to the vein's opening at the skin. This helps to avoid accidental exposure of the patient's skin at the incision or elsewhere to the fill power of the laser beam being used to close the vein. Also attending hospital personnel such as nurses, surgeons, anesthesiologists, are protected from exposure to the laser beam energy.

Another preferred embodiment is depicted in FIG. 3. Spiral pattern 302 frequency increases as it reaches distal end of introducer sheath 300. This allows the surgeon to be warned of closeness to distal end and can act accordingly to prevent accidental exposure of laser energy to patient's skin or other attending hospital personnel.

In another preferred embodiment spiral pattern may vary in color or hue to indicate relative distance of fiber tip to the vein's opening at the skin. For example, green spiral pattern changes to yellow and finally to red at a few centimeters from distal end.

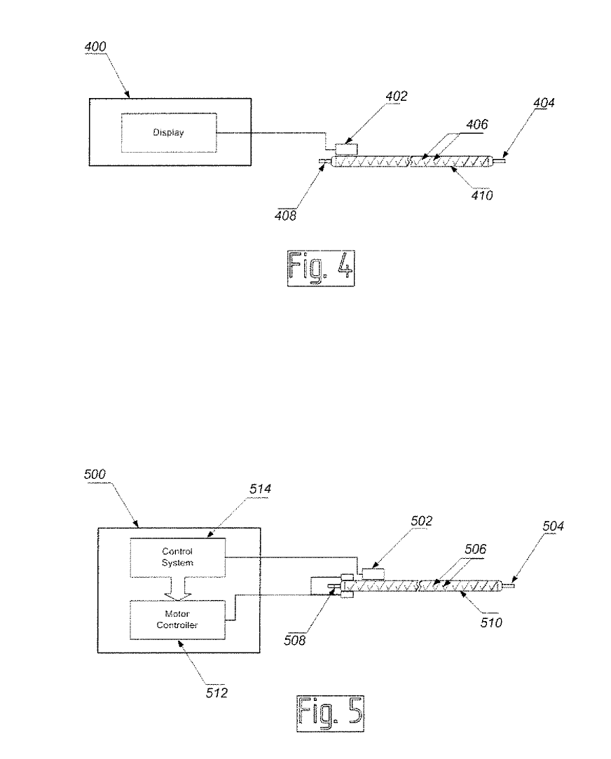

FIG. 4 shows another example that includes manual withdrawal with a reader to monitor speed of withdrawal to help surgeon maintain his desired pull back speed. In this embodiment, initial positioning of fiber 408 is done manually with the fiber tip 404 exposed a few centimeters past the distal end of catheter/introducer 410. Then the proximal end of the fiber 408 and catheter/introducer 410 are engaged together. When pulling back, top spiral marks 406 are read by optical sensor 402 giving information to the surgeon to move fiber and catheter 410 to a determined position at a determined speed.

In another embodiment, as shown in FIG. 5, initial positioning of fiber 508 is done manually with the fiber tip 504 exposed a few centimeters past the tip of catheter/introducer 510. Then the proximal end of fiber 508 and catheter/introducer 510 are engaged in or attached to a holder attached to step motor 512. The joined section is withdrawn at a set speed by automatic pull-back device 500; Top spiral marks 506 are read by optical sensor 502 giving information to control system 514 that drives step motor 512 to move fiber and catheter/introducer 510 to a determined position at a determined speed. Optical reader maybe such as described in co-owned application Ser. No. 11/443,143.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A specially marked device for accurate insertion, positioning and withdrawal of an optical fiber or introducer during minimally invasive laser treatment.

2. The specially marked device according to claim 1, wherein said minimally invasive laser treatment comprises tracheal treatment, esophagus treatment or greater saphenous vein treatment.

3. The specially marked device according to claim 1, wherein said specially marked device comprises an introducer, an optical fiber or a jacketed optical fiber.

4. The specially marked device according to claim 3, wherein the special marking comprises a mark in the form of a continuous spiral on an outer surface of said introducer, said optical fiber or said optical fiber jacket.

5. The specially marked device according to claim 4, wherein said special marking further comprises at least two position marks on said outer surface of said optical fiber, wherein said at least two position marks are at the proximal end of said optical fiber, one mark indicating alignment of fiber tip with the distal end of said introducer, the second mark indicating position of fiber tip with said introducer is applicable to start lasing.

6. The specially marked device according to claim 4, wherein said spiral has a change in its properties as it approaches a distal end of the surface it is on.

7. The specially marked device according to claim 6, wherein said change in properties of said spiral is to make, at the distal end, a circle with said spiral changing a property selected from the group consisting of a broadening of the spiral, an increase in the spiral pattern frequency, a color change in the spiral, a shading or hue enhancement of the spiral.

8. The specially marked device according to claim 1, wherein said specially marked device further comprises an optical sensor.

9. The specially marked device according to claim 8, wherein said optical sensor uses said continuous spiral mark to determine position of said optical fiber and said introducer.

10. The specially marked device according to claim 8 wherein said specially marked device further comprises a holder, a step motor, an automatic pull back device and a control system.

11. The specially marked device according to claim 10, wherein the proximal end of said optical fiber and the proximal end of said introducer are attached to said holder.

12. The specially marked device according to claim 10, wherein said holder is attached to said step motor.

13. A procedure for accurate insertion, positioning and withdrawal of an optical fiber or introducer during minimally invasive laser treatment using the specially marked device of claim 1, comprising the steps of:
insert said specially marked device to a treatment site using at least two position marks to appropriately position said specially marked device, and;
apply laser energy while pulling back on said specially marked device at a predetermined speed, using continuous spiral mark on outer surface of said specially marked device to determine relative position and speed of withdrawal of said specially marked device.
